(19) United States
(12) Patent Application Publication
(10) Pub. No.: US 2025/0157056 A1
(43) Pub. Date: May 15, 2025

(54) SYSTEMS AND METHODS OF CORRECTING MOTION IN IMAGES FOR RADIATION PLANNING

(71) Applicant: Washington University, St. Louis, MO (US)

(72) Inventors: Geoffrey HUGO, St. Louis, MO (US); Phillip CUCULICH, St. Louis, MO (US); Clifford ROBINSON, St. Louis, MO (US)

(21) Appl. No.: 18/837,721

(22) PCT Filed: Feb. 15, 2023
(86) PCT No.: PCT/US2023/062634

§ 371 Date: Aug. 12, 2024

Related U.S. Application Data
(60) Provisional application No. 63/314,798, filed on Feb. 28, 2022.

(51) Int. Cl. G06T 7/246; A61N 5/10; G06T 7/00

(52) U.S. Cl. CPC G06T 7/251; A61N 5/1037; G06T 7/0012; G06T 2207/10076; G06T 2207/20084; G06T 2207/30048

(57) ABSTRACT

A computer-implemented method of correcting motion in respiratory four-dimensional computed tomography (4DCT) images of a subject in radiation planning is provided. The method includes receiving respiratory 4DCT images, wherein the respiratory 4DCT images were acquired while a subject was free breathing. The method also includes receiving cardiac four dimensional (4D) images of the subject, wherein the cardiac 4D images were acquired while the subject was in breath hold. The method further includes deriving a cardiac motion model based on the cardiac 4D images, wherein the cardiac motion model includes frames of images of the subject, each frame corresponding to a cardiac phase in a cardiac cycle, each frame of images including motion fields at the cardiac phase. The method further includes correcting motion in the respiratory 4DCT images using the cardiac motion model, and outputting the corrected respiratory 4DCT images.

### CROSS-REFERENCE TO RELATED APPLICATIONS

This patent application claims the benefit of U.S. Provisional Patent Application No. 63/314,798, filed on Feb. 28, 2022, titled “SYSTEMS AND METHODS OF CORRECTING MOTION IN IMAGES FOR RADIATION PLANNING,” the entire contents and disclosures of which are hereby incorporated herein by reference in their entirety.

### BACKGROUND

The field of the disclosure relates generally to systems and methods of medical image processing, and more particularly, to systems and methods of correcting motion in images used for radiation planning.

Ventricular tachycardia is a life threatening illness, which may be treated with radioablation. The effectiveness of radioablation is affected by target precision of radiation because radiation is nonselective and may also harm healthy tissue from an unsatisfactory precision. CT (computed tomography) images are used in radiation planning. Motion, such as respiratory and heartbeat motion, in these images renders them unsuitable in precision radiation planning.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

In one aspect, a computer-implemented method of correcting motion in respiratory four-dimensional computed tomography (4DCT) images of a subject in radiation planning is provided. The method includes receiving respiratory 4DCT images, wherein the respiratory 4DCT images were acquired while a subject was free breathing. The method also includes receiving cardiac four dimensional (4D) images of the subject, wherein the cardiac 4D images were acquired while the subject was in breath hold. The method further includes deriving a cardiac motion model based on the cardiac 4D images, wherein the cardiac motion model includes frames of images of the subject, each frame corresponding to a cardiac phase in a cardiac cycle, each frame of images including motion fields at the cardiac phase. The method further includes correcting motion in the respiratory 4DCT images using the cardiac motion model, and outputting the corrected respiratory 4DCT images.

In another aspect, a radiation planning system is provided. The system includes a computing device, the computing device including at least one processor in communication with at least one memory device. The at least one processor is programmed to receive respiratory 4DCT images, wherein the respiratory 4DCT images were acquired while a subject was free breathing. The at least one processor is also programmed to receive cardiac 4D images of the subject, wherein the cardiac 4D images were acquired while the subject was in breath hold. The at least one processor is further programmed to derive a cardiac motion model based on the cardiac 4D images, wherein the cardiac motion model includes frames of images of the subject, each frame corresponding to a cardiac phase in a cardiac cycle, each frame of images including motion fields at the cardiac phase. In addition, the at least one processor is programmed to correct motion in the respiratory 4DCT images using the cardiac motion model and output the corrected respiratory 4DCT images.

### DRAWINGS

The patent or application file contains at least one drawing executed in color. Copies of this patent or patent application publication with color drawing(s) will be provided by the Office upon request and payment of the necessary fee.

These and other features, aspects, and advantages of the present disclosure will become better understood when the following detailed description is read with reference to the accompanying drawings in which like characters represent like parts throughout the drawings, wherein:

FIG. 1A is a flow chart of an example method of motion correction.

FIG. 1B is a schematic diagram of an example radiation planning system.

FIG. 2A is a schematic diagram of a neural network model.

FIG. 2B is a schematic diagram of a neuron in the neural network model shown in FIG. 2A.

FIG. 3 is a block diagram of an example computing device.

FIG. 4A is a schematic diagram of a radiotherapy.

FIG. 4B shows radiation dose distribution from cardiac radioablation treatment.

FIG. 4C shows efficacy of the radioablation treatment.

FIG. 4D shows median ventricular tachycardia (VT) events before and after radioablation treatment.

FIG. 5A shows survival rates stratified by planning target volume.

FIG. 5B shows the impact of target volume on dose to non-target tissue.

FIG. 6A is a comparison of a respiratory image and a cardiac image.

FIG. 6B shows improvement in corrected respiratory images of a phantom using the systems and methods described herein.

FIG. 6C shows underestimation of heart motion of the phantom when using a known method.

FIG. 7A is a schematic diagram of correcting motion in respiratory images.

FIG. 7B is a schematic diagram of groupwise registration.

FIG. 7C is a schematic diagram of reducing artifacts in respiratory images.

FIG. 8 shows a visual feedback breathing guidance system.

FIG. 9A is a schematic diagram of segmenting images to include anatomical segments.

FIG. 9B is a comparison of segmentation by a neural network model with manual segmentation as the ground truth.

FIG. 9C is a plot showing a comparison between the contours of segments generated by a neural network model and segments manually annotated.

FIG. 10 is a schematic diagram of computing cumulative dose.

### DETAILED DESCRIPTION

The disclosure includes systems and methods of correcting motion of respiratory images used in radiation planning of a subject. As used herein, a subject is a human, an animal, or a phantom, or part of the human, the animal, or the phantom. A phantom may be a physical phantom or a computational phantom. The method aspects will be in part apparent and in part explicitly discussed in the following description.

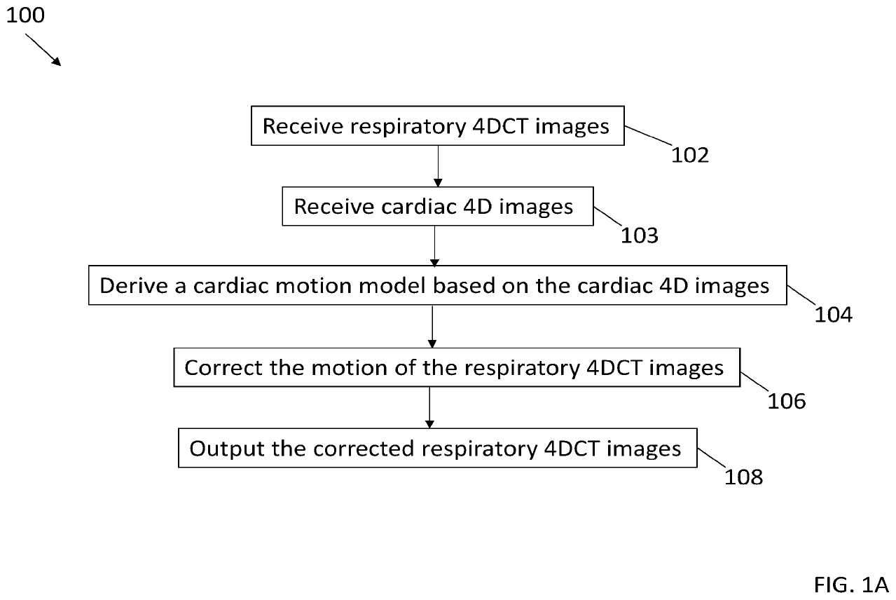

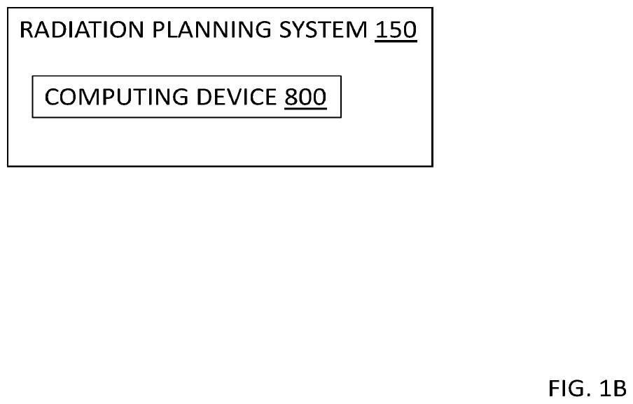

FIG. 1A is a flow chart of an example method 100 of correcting motion of respiratory images of a subject. In the example embodiment, the method 100 includes receiving 102 respiratory images. As used herein, respiratory images are a series of images of the subject acquired while the subject was free breathing. The respiratory images are four dimensional (4D), which are a series of images of a three-dimensional (3D) volume in the subject, with the fourth-dimension of the images being time. The images may be acquired by CT. For radiation planning purpose, the images are typically acquired using a CT scanner because CT images have information on electron density information and can be used to calculated radiation dosage. Respiratory images are used for radiation planning to match the anatomy during radiation also because radiation therapy is performed while the subject is free breathing. 4D respiratory images acquired by CT may be referred to as respiratory 4DCT images.

In the example embodiment, the method 100 further includes receiving 103 cardiac 4D images of the subject. As used herein, cardiac images are a series of images of the subject acquired while the subject was in breath hold. The cardiac images may be in 4D as a series of images of a volume in the subject with the fourth dimension being time. Cardiac images may be acquired by CT or other modalities such as magnetic resonance imaging (MRI). Cardiac images may be acquired at the same imaging session with the respiratory images. In some embodiments, cardiac images are acquired at different time from the respiratory images or using a different CT scanner or different modalities such as MRI. 4D cardiac images acquired by CT may be referred to as cardiac 4DCT images. Because cardiac images are acquired while the subject is in breath hold, the effects of respiratory motion on the images are reduced. As such, the image quality of cardiac images are less affected by respiratory motion and is better than that of respiratory images. Further, cardiac images may be acquired to have higher temporal and/or spatial resolution than respiratory images. The cardiac images and the respiratory images are images of the same anatomy, or at least having overlapping anatomies such that respiratory images may be registered to the cardiac images to correct motion in respiratory images. For example, both respiratory images and cardiac images are images of a heart and/or surrounding organs of the subject. Therefore, cardiac images are used to improve the accuracy of dosage calculation for radiation planning by registering respiratory images with cardiac images.

In the example embodiment, the method 100 further includes deriving 104 a cardiac motion model based on the cardiac 4D images. As used herein, a cardiac motion model includes frames of images, each frame of images corresponding to a cardiac phase in a cardiac cycle and having a motion field associated with each voxel in the images that indicate the motion of that voxel at that cardiac phase. A motion model may be used to estimate motion fields between frames or phases by interpreting the motion fields at the intermediate time point between adjacent phases using motion fields at the adjacent phases. A motion field or a motion vector field is a vector field with each vector in the field indicating motion at that voxel, where the magnitude of the vector indicates the magnitude of the motion and the direction of the vector indicates the direction of the motion. A frame is an image of a plane or images of a volume at a point of time in a cardiac or respiratory cycle, such as a cardiac phase in a cardiac cycle or a respiratory phase in a respiratory cycle. The images may be in 2D or 3D. 4DCT images or 4D images acquired by other modalities include a series of frames of 3D images of a volume. The fourth dimension of the 4D images may be time, a cardiac phase in a cardiac cycle, or a respiratory phase in a respiratory cycle. An example number of frames is 10. The cardiac 4D images may be rebinned or rearranged according to the cardiac phases of the images to generate frames of cardiac images, with each frame corresponding to a cardiac phase.

In the example embodiment, the method 100 also include correcting 106 the motion of the respiratory 4DCT images. The respiratory images maybe rebinned or rearranged according to the respiratory phases of the images to generate frames of respiratory images, with each frame corresponding to a respiratory phase. As used herein, frames of respiratory images are series of respiratory images of the entire slice coverage or portions of the slice coverage (referred to as stacks of respiratory images as described below) at corresponding respiratory phases. In order to correct the respiratory images using the cardiac motion model, a corresponding cardiac phase in the cardiac motion model for each frame of respiratory images is identified. If the respiratory images are acquired simultaneously with ECG signals, the corresponding cardiac phase of each frame of respiratory images is identified using the ECG signals by locating the cardiac phase of the frame of respiratory images in the cardiac cycle based on the ECG signals. If ECG signals are not acquired during the acquisition of respiratory images, the corresponding phase is determined by the image similarity of the frame of respiratory images with each phase in the cardiac motion model. Image similarity may be measured using indexes such as structural similarity index measure indexes. The phase in the cardiac motion model that is most similar to the respiratory images is selected as the cardiac phase for that frame of respiratory images. The selection of the corresponding cardiac phase is repeated for each frame of respiratory images.

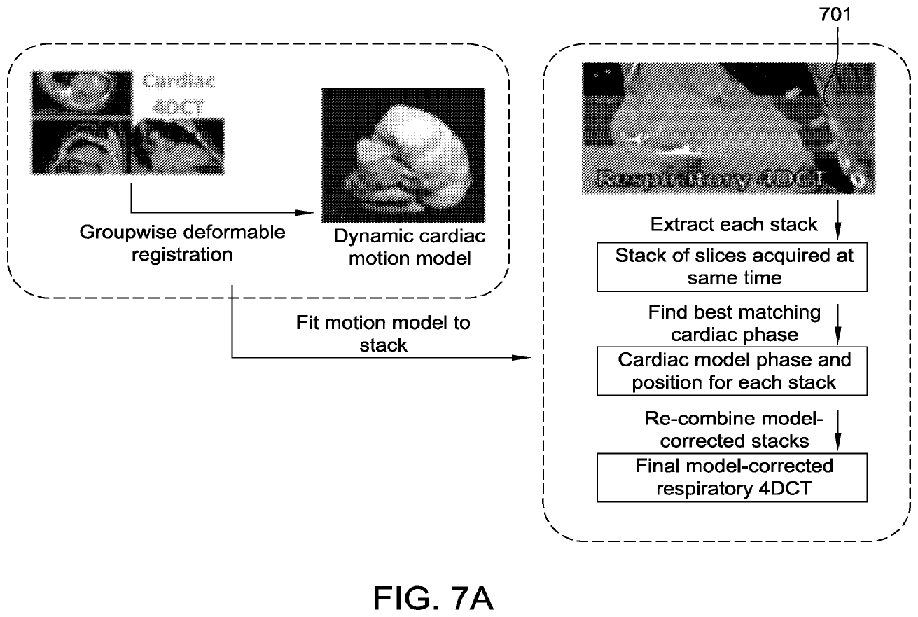

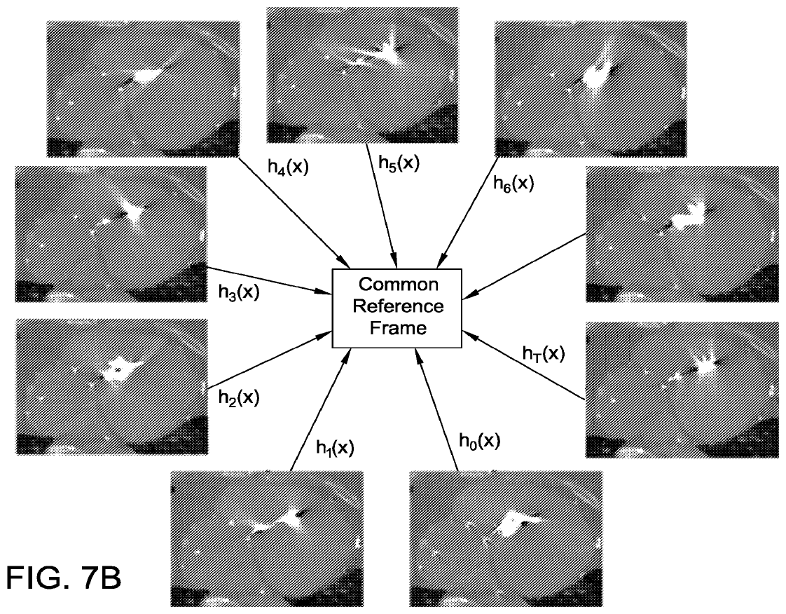

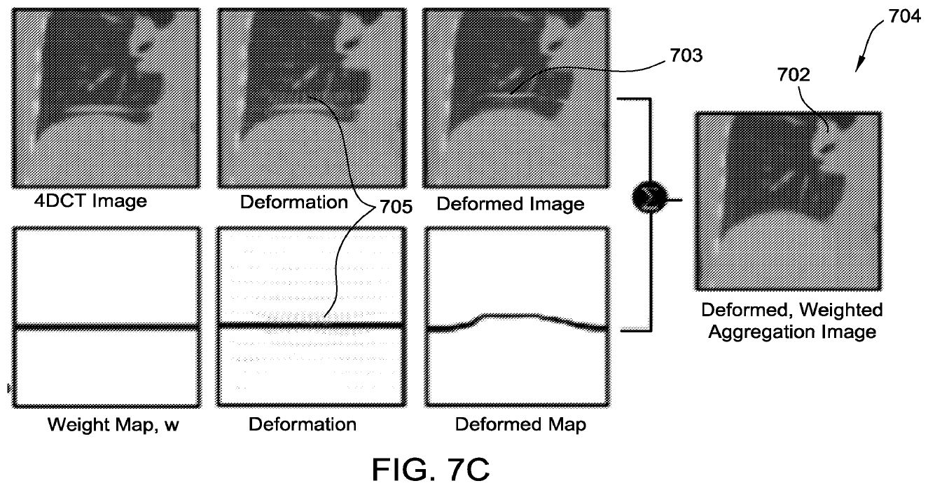

In the example embodiments, the respiratory images are stacks of respiratory images, each stack covering a portion of a slice coverage (see, e.g., the stacks 701 marked in FIG. 7A). The cardiac phase of a stack of respiratory images is determined similar to the mechanisms in determining the cardiac phase of a frame of respiratory images. For example, if the respiratory images are acquired simultaneously with ECG signals, the phase of a stack is determined as the cardiac phase of the stack in a cardiac cycle based on the ECG signals. If ECG signals are not acquired during the acquisition of respiratory images, the stack of respiratory images are compared with each phase in the cardiac motion model. The phase that has images most similar to the stack of respiratory images is selected as the cardiac phase of the stack.

In the example embodiment, once the corresponding cardiac phase of the respiratory images is determined, the cardiac motion model is used to correct 106 the motion of the respiratory images, by using the motion fields in the cardiac motion model at the determined cardiac phase as parameters of transformation to correct motion. Stacks of corrected respiratory images may be combined to form corrected respiratory images by stacking the stacks together, and generate frames of corrected respiratory images, where each frame covers the entire slice coverage.

In the example embodiment, artifacts in the corrected respiratory images may be reduced by generating a weight map corresponding to outliers across frames of the cardiac images at the same voxels. The artifacts may be reduced by downweighing or dividing the corrected respiratory images at each voxel with the weight in the weight map at the voxel. Image quality may be further increased by groupwise registering all frames of cardiac images, respiratory images, or stacks of respiratory images with a common reference frame. Groupwise registration may be combined with down weighting of artifacts.

If the cardiac images are acquired during the same session as the respiratory images, the imaged anatomy is the same, and rigid registration between cardiac images and respiratory images may be used to correct motion in respiratory images. As used herein, rigid registration refers to image registration using linear transformation models such as rotation, translation, and/or scaling. If the cardiac images are acquired at different time, the imaged anatomy may have changed. Similarly, if the cardiac images are acquired with a different modality, such as MRI, from CT used for acquiring respiratory images, the anatomy appeared in the respiratory images are also different from that in the cardiac images, for example, due to different modality having different imaging contrast mechanisms. As such, rigid registration between cardiac images and respiratory images to correct the motion of respiratory images may fail.

To solve this problem, the cardiac images and the respiratory images are segmented in the same way, such as AHA (American Heart Association) 17 segments used in clinic. The segments may be derived using a neural network model, using a method not based on a neural network model such as principal components analysis, using a manual method such as manually annotating by a clinician, or any combination thereof. For example, anatomical features such as left and right ventricles, atria, vales, and/or left anterior descending coronary artery may be auto-segmented using a neural network model. Anatomical keypoint features such as interventricular grooves and the long axis of the left ventricle are derived based on the anatomical features, using methods such as principal components analysis. Anatomical keypoint features are used to map a standardized segment model, such as the standardized myocardial segmentation model of the AHA 17 segment model having standardized myocardial segments, onto the left ventricle of each image in the cardiac images, respiratory images, or stacks of respiratory images. Although the anatomy of the subject may appear different in different imaging sessions and/or different modalities, the anatomical features identified by the AHA 17 segment model remain relatively unchanged. The mapped segment models are used to guide the registration process, where the segment model identifies the corresponding segments in each frame and the identified corresponding segments are registered in derive cardiac motion model and improve image quality of respiratory images. The segment-based registration described herein is advantageous in improving the image quality because the segments used in the registration are anatomical segments of a subject, which are more reliable features for registration than typical segments of an image which may change in different imaging sessions or modalities.

In the example embodiment, the method 100 further includes outputting 108 the corrected respiratory images. The corrected respiratory images may be used in determining the dosage for radiation planning.

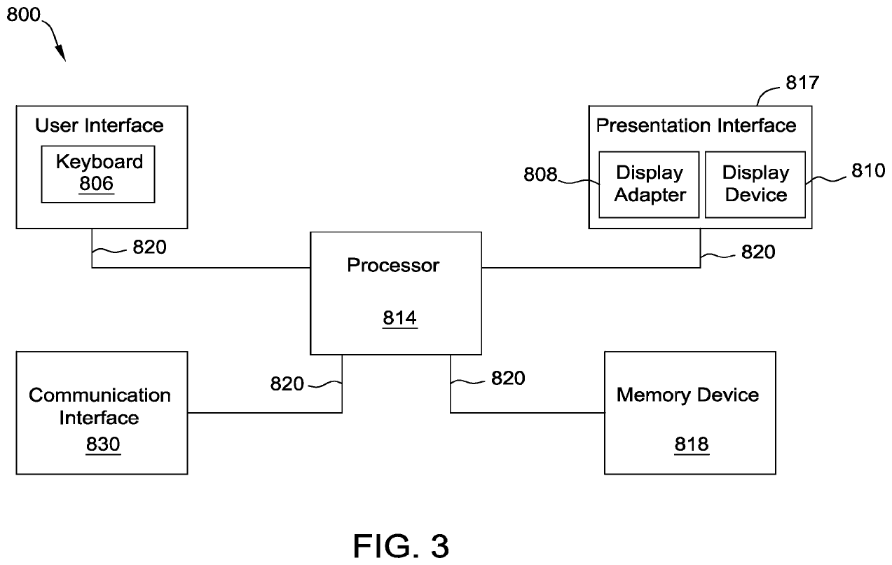

FIG. 1B is a schematic diagram of an example radiation planning system 150. Radiation planning system 150 includes a computing device 800 (see FIG. 3 described later). Computing device 800 is configured to correct motion in respiratory 4DCT images. Method 100 may be implemented on computing device 800. Method 100 may be implemented on multiple computing devices 800 in communication with one another or on one computing device 800. Computing device 800 may be a server computing device.

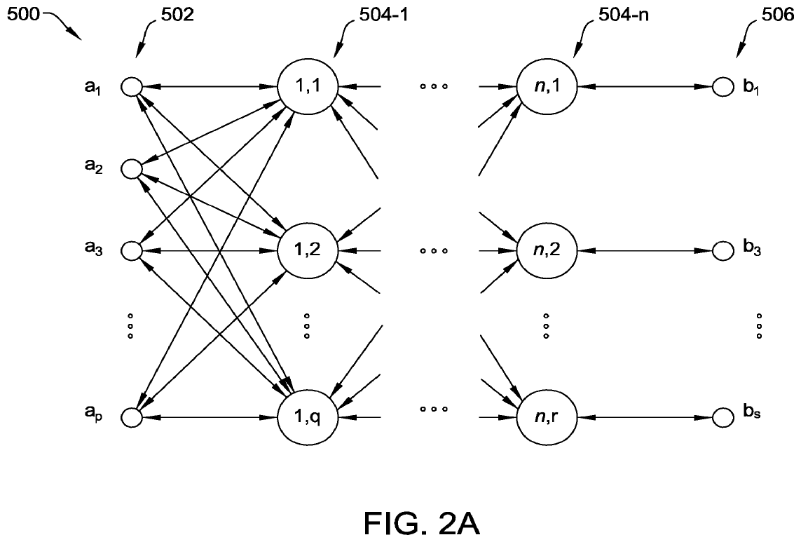

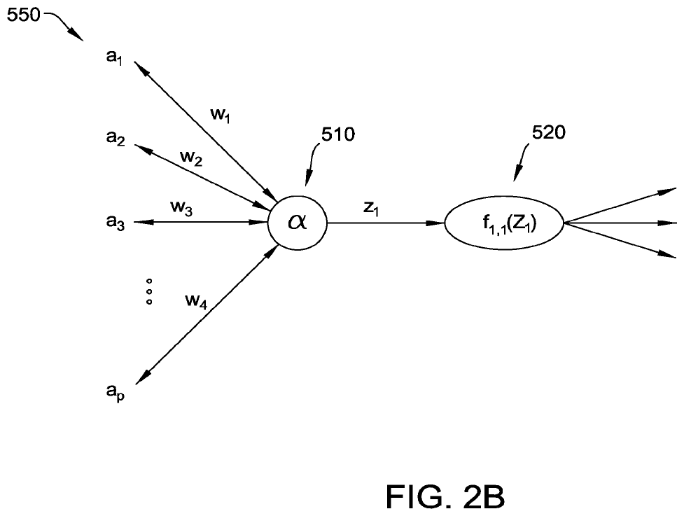

FIG. 2A depicts an example artificial neural network model 204 that may be used in the systems and methods described herein. The example neural network model 204 includes layers of neurons 502, 504-1 to 504-n, and 506, including an input layer 502, one or more hidden layers 504-1 through 504-n, and an output layer 506. Each layer may include any number of neurons, i.e., q, r, and n in FIG. 2A may be any positive integers. It should be understood that neural networks of a different structure and configuration from that depicted in FIG. 2A may be used to achieve the methods and systems described herein.

In the example embodiment, the input layer 502 may receive different input data. For example, the input layer 502 includes a first input a₁ representing training images, a second input a₂ representing patterns identified in the training images, a third input a₃ representing edges of the training images, and so on. The input layer 502 may include thousands or more inputs. In some embodiments, the number of elements used by the neural network model 204 changes during the training process, and some neurons are bypassed or ignored if, for example, during execution of the neural network, they are determined to be of less relevance.

In the example embodiment, each neuron in hidden layer(s) 504-1 through 504-n processes one or more inputs from the input layer 502, and/or one or more outputs from neurons in one of the previous hidden layers, to generate a decision or output. The output layer 506 includes one or more outputs each indicating a label, confidence factor, weight describing the inputs, and/or an output image. In some embodiments, however, outputs of the neural network model 204 are obtained from a hidden layer 504-1 through 504-n in addition to, or in place of, output(s) from the output layer(s) 506.

In some embodiments, each layer has a discrete, recognizable function with respect to input data. For example, if n is equal to 3, a first layer analyzes the first dimension of the inputs, a second layer the second dimension, and the final layer the third dimension of the inputs. Dimensions may correspond to aspects considered strongly determinative, then those considered of intermediate importance, and finally those of less relevance.

In other embodiments, the layers are not clearly delineated in terms of the functionality they perform. For example, two or more of hidden layers 504-1 through 504-n may share decisions relating to labeling, with no single layer making an independent decision as to labeling.

FIG. 2B depicts an example neuron 550 that corresponds to the neuron labeled as “1,1” in hidden layer 504-1 of FIG. 2A, according to one embodiment. Each of the inputs to the neuron 550 (e.g., the inputs in the input layer 502 in FIG. 2A) is weighted such that input a₁ through aₚ corresponds to weights w₁ through wₚ as determined during the training process of the neural network model 204.

In some embodiments, some inputs lack an explicit weight, or have a weight below a threshold. The weights are applied to a function a (labeled by a reference numeral 510), which may be a summation and may produce a value z₁ which is input to a function 520, labeled as ƒ_{1,1}(z₁). The function 520 is any suitable linear or non-linear function. As depicted in FIG. 2B, the function 520 produces multiple outputs, which may be provided to neuron(s) of a subsequent layer, or used as an output of the neural network model 204. For example, the outputs may correspond to index values of a list of labels, or may be calculated values used as inputs to subsequent functions.

It should be appreciated that the structure and function of the neural network model 204 and the neuron 550 depicted are for illustration purposes only, and that other suitable configurations exist. For example, the output of any given neuron may depend not only on values determined by past neurons, but also on future neurons.

The neural network model 204 may include a convolutional neural network (CNN), a deep learning neural network, a reinforced or reinforcement learning module or program, or a combined learning module or program that learns in two or more fields or areas of interest. Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. The neural network model 204 may be trained using unsupervised machine learning programs. In unsupervised machine learning, the processing element may be required to find its own structure in unlabeled example inputs. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, object statistics, and information. The machine learning programs may use deep learning algorithms that may be primarily focused on pattern recognition, and may be trained after processing multiple examples. The machine learning programs may include Bayesian Program Learning (BPL), voice recognition and synthesis, image or object recognition, optical character recognition, and/or natural language processing-either individually or in combination. The machine learning programs may also include natural language processing, semantic analysis, automatic reasoning, and/or machine learning.

Based upon these analyses, the neural network model 204 may learn how to identify characteristics and patterns that may then be applied to analyzing image data, model data, and/or other data. For example, the model 204 may learn to identify features in a series of data points.

Systems and methods described herein may be implemented on any suitable computing device 800 and software implemented therein. FIG. 3 is a block diagram of an example computing device 800. In the example embodiment, the computing device 800 includes a user interface 804 that receives at least one input from a user. The user interface 804 may include a keyboard 806 that enables the user to input pertinent information. The user interface 804 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the example embodiment, computing device 800 includes a display interface 817 that presents information, such as input events and/or validation results, to the user. The display interface 817 may also include a display adapter 808 that is coupled to at least one display device 810. More specifically, in the example embodiment, the display device 810 may be a visual display device, such as a cathode ray tube (CRT), a liquid crystal display (LCD), a light-emitting diode (LED) display, and/or an “electronic ink” display. Alternatively, the display interface 817 may include an audio output device (e.g., an audio adapter and/or a speaker) and/or a printer.

The computing device 800 also includes a processor 814 and a memory device 818. The processor 814 is coupled to the user interface 804, the display interface 817, and the memory device 818 via a system bus 820. In the example embodiment, the processor 814 communicates with the user, such as by prompting the user via the display interface 817 and/or by receiving user inputs via the user interface 804. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are example only, and thus are not intended to limit in any way the definition and/or meaning of the term “processor.”

In the example embodiment, the memory device 818 includes one or more devices that enable information, such as executable instructions and/or other data, to be stored and retrieved. Moreover, the memory device 818 includes one or more computer readable media, such as, without limitation, dynamic random access memory (DRAM), static random access memory (SRAM), a solid state disk, and/or a hard disk. In the example embodiment, the memory device 818 stores, without limitation, application source code, application object code, configuration data, additional input events, application states, assertion statements, validation results, and/or any other type of data. The computing device 800, in the example embodiment, may also include a communication interface 830 that is coupled to the processor 814 via the system bus 820. Moreover, the communication interface 830 is communicatively coupled to data acquisition devices.

In the example embodiment, the processor 814 may be programmed by encoding an operation using one or more executable instructions and providing the executable instructions in the memory device 818. In the example embodiment, the processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

### Examples

### A. Significance

### A.1. Ventricular Tachycardia—a Life Threatening Illness

Sudden cardiac arrest is the single largest cause of death in the developed world, and causes over 325,000 deaths per year in the US; more than lung cancer, breast cancer and AIDS combined. Ventricular tachycardia (VT) is a life-threatening heart rhythm and the most common cause of sudden cardiac arrest. The underlying cause of VT is development of abnormal electrical circuits within scar tissue inside myocardium, either from a previous myocardial infarction or a non-ischemic cardiomyopathy. Current treatment for VT includes medications, implantable cardiac defibrillator (ICD) placement, and catheter ablation. Medications used for treatment of VT, such as amiodarone and mexilitine, have considerable acute and late toxicities, are poorly tolerated by patients, and have poor durable arrhythmia control. Placement of an ICD allows for acute disruption of VT through anti-tachycardia pacing or shocks, but at the expense of reduced quality of life or further decline in cardiac function from continuous shocks. Often, these interventions are insufficient to effectively control VT on their own. Worldwide, catheter ablation is increasingly performed to treat VT. While the procedure is especially effective in the absence of ventricular scar, recurrence rates as high as 50% at 6 months are seen in the setting of cardiomyopathy VT. Recurrences are more common because of a larger scar size, which leads to: 1) multiple coexisting VT circuits; 2) circuits that extend deep within the myocardium, outside the reach of standard catheter ablation energy; 3) formation of new circuits after the ablation procedure. Recurrent VT after catheter ablation is associated with 5 to 10-fold higher odds of death, often due to incomplete ablation. Further, many patients will not be candidates for catheter ablation due to age, comorbidities, or technical inability to safely ablate the region of interest. Thus, many patients will ultimately develop irreversible debilitating refractory VT.

### A.2. Cardiac Radioablation for Treatment of VT

Given the limitations of currently available treatment modalities for patients with treatment refractory ventricular arrhythmia, there is a compelling need to a develop safer, more effective therapy. In principle, this would be best achieved by 1) improving the quality of the ablation (gap-free, full myocardial thickness) while 2) mitigating the high risks of invasive procedures. An attractive alternative to invasive catheter ablation is cardiac radioablation, first developed through a small number of clinical case studies and preclinical studies. Cardiac radioablation includes the combination of multi-modality imaging and electroanatomical information to guide the delivery of stereotactic ablative body radiotherapy, a highly precise form of radiotherapy used to treat small, localized targets such as brain, lung, and liver tumors and metastases.

A pilot program was initiated to explore treatment of patients with high risk, refractory VT with a combination of noninvasive cardiac mapping and noninvasive radioablation. In cardiac radioablation, patients underwent pre-treatment targeting by using available cardiac scar imaging and electroanatomical mapping. The data was converted into a target on the radioablation planning CT and a single dose of 25 Gy was then delivered using standard stereotactic ablative body radiotherapy (SABR) techniques on a linear accelerator with on-board image guidance. Five patients with high risk, treatment-refractory VT underwent treatment. Mean noninvasive VT ablation time was 14 minutes (range 11-18 minutes), performed awake. In total, there was a >99% reduction in total VT burden (6577 ICD therapies in the 3 months before treatment to 4 ICD therapies in the 12 months after treatment). Reduction in VT burden was achieved in all patients (mean 1315 per-patient ICD therapies (range 5-4312) being reduced to 1 (range 0-2)) despite stopping antiarrhythmic medication. Cardiac function (left ventricular ejection fraction) did not adversely change over time. Mild adjacent lung inflammatory changes were observed at 3 months, which resolved by one year.

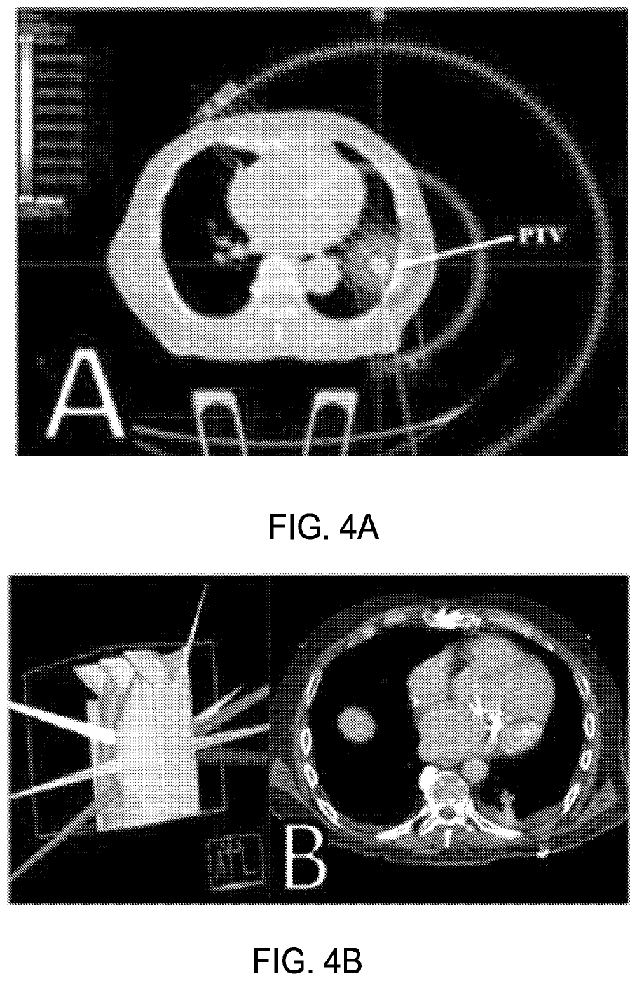

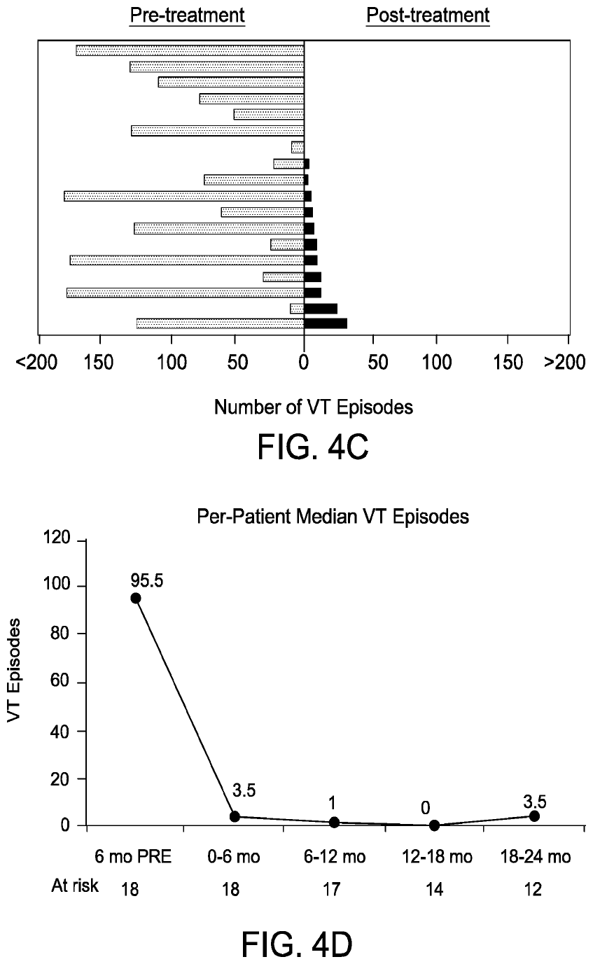

A phase I/II trial of cardiac radioablation (ENCORE-VT) was conducted. Nineteen subjects with high-risk refractory VT were enrolled who previously underwent a median of 1 VT catheter ablation procedures (range 0-4) and with 59% on >1 anti-arrhythmic drug. FIGS. 4A-4D show cardiac radioablation clinical implementation and results. FIG. 4A is a schematic diagram of stereotactic ablative body radiotherapy (SABR) treatment for lung cancer. Multiple beams are precisely arranged to deliver a compact, conformal dose to the target allowing for safe delivery of high doses while sparing dose to surrounding non-target tissues. FIG. 4B shows radiation dose distribution from cardiac radioablation treatment. FIG. 4C is a plot of efficacy, in 19 patients, ENCORE-VT phase I/II trial where total ICD therapies per patient 6 month before are depicted with blue bars (1782 VT events) and after are depicted with red bars (111 VT events). FIG. 4D shows VT reduction is durable, as shown by median VT events over 24 months. Gross ablation volume was 25.4 cc (range 6.4-88.6) delivered in a single treatment with median duration of 15.3 minutes (range 5.4-32.3), while the patient was awake. Overall number of VT events (FIGS. 4A-4D) from 6 months before and after radioablation was markedly reduced (1782 vs. 111 VT events, 94% reduction). Median number of VT events per patient before and after radioablation was 119 vs. 3 (p<0.001). Use of dual antiarrhythmic medication decreased from 59% to 12% (p=0.008) and significant improvements were observed in SF-36 quality of life measures.

### A.3. Challenges to Clinical Deployment of Cardiac Radioablation

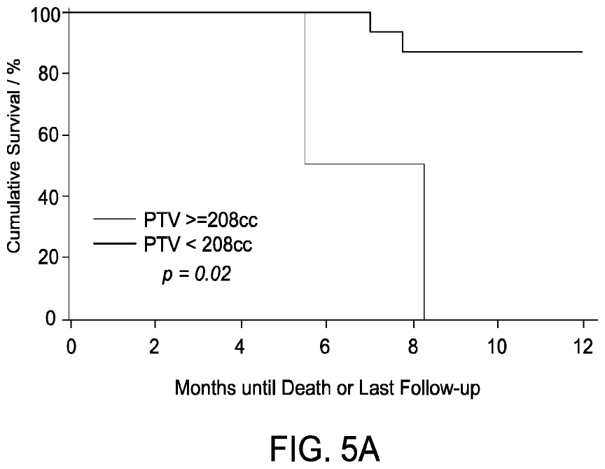

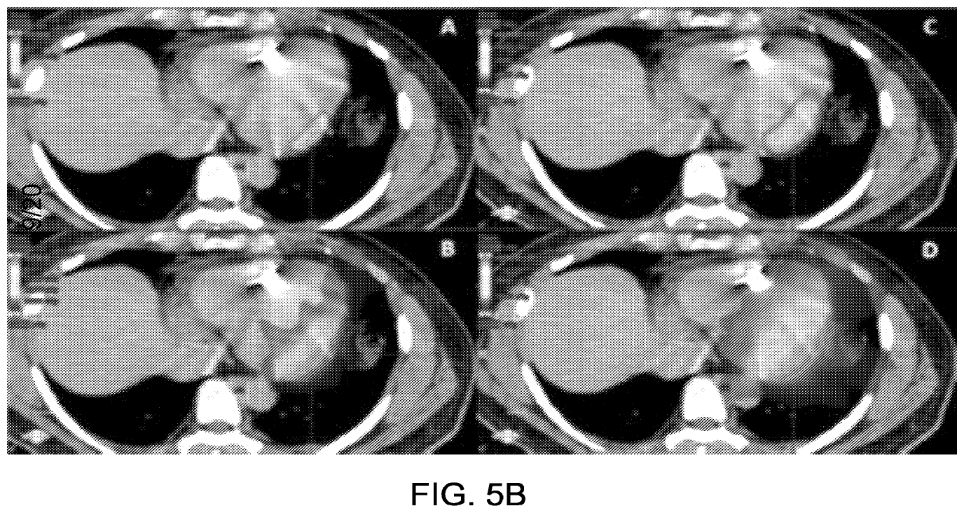

As in stereotactic ablative body radiotherapy, for cardiac radioablation the cognitive treatment design work, or treatment planning, occurs up front and includes virtual construction and assessment of the planned radiation delivery. Despite the promising early clinical results described above, it has become clear that current cardiac radioablation imaging and treatment planning techniques will not be suitable for widespread deployment to the community and treatment of the wider population of patients. The philosophy in early trials and case studies has been to treat conservatively, irradiating a target with a substantial safety margin to ensure, despite uncertainty in imaging and planning, that the target is not missed. However, this results in larger target volumes, which as shown in FIGS. 5A and 5B are associated with a higher risk of mortality. Furthermore, larger targets lead to an increased risk of toxicity or may limit patients eligible for treatment.

FIG. 5A shows overall survival in ENCORE-VT stratified by planning target volume. The blue line is for volume greater or equal to 208 cc, and the red line is for volume less than 208 cc. FIG. 5B shows the impact of target volume on dose to non-target tissue. Color wash shows radiation dose, which is thresholded at 25 Gy (A and C) or 10 Gy (B and D). In A and B, 25 Gy is prescribed to a target with minimal margin for error, while the target in C and D includes a more conservative margin. Note the significantly increased dose to non-target heart and lung with treatment to a larger target. In C and D, mean heart dose is 3.3 Gy, and is 1.7 Gy in A and B, which represents an estimated 50% decrease in relative risk of cardiac events.

In contrast, high spatial and temporal resolution multi-modality motion models for cardiac radioablation treatment planning will enable high precision (<3 mm overall error) planning and delivery. In the long term, this will lead to better efficacy and toxicity prediction, reduced cardiotoxicity, and better availability of cardiac radioablation to more patients. The following three key problems will be solved, enabling effective translation of cardiac radioablation into the community:

### A.3.1. Lack of Available 4D Imaging to Capture Simultaneous Cardiac and Respiratory Motion

Current cardiac and respiratory-correlated 4DCT techniques are not designed to measure motion of structures, such as the heart, undergoing cardiac motion during free breathing. Cardiac 4D imaging is widely available on modern CT scanners, with electrocardiogram (ECG)-correlated scans routinely being acquired with the patient in breath hold. Cardiac 4D imaging may be ECG triggered, where the acquisition of images is triggered by ECG signals. Cardiac 4D imaging may not ECG triggered, where ECG signals are used retrospectively to rearrange the images according to the cardiac phases of the images. Similarly, respiratory-correlated 4DCT emerged in the early 2000s for radiotherapy, using a respiratory signal to sort projection or image data into bins. However, as heartbeat and respiration are generally uncorrelated, no current imaging technique may simultaneously capture high frequency 3D frames of both cardiac and free-breathing respiratory motion of the heart. Cardia-correlated 4DCT may be referred to as cardiac 4DCT. Respiratory-correlated 4DCT may be referred to as respiratory 4DCT. Respiratory 4DCT images are typically acquired while the subject is free breathing.

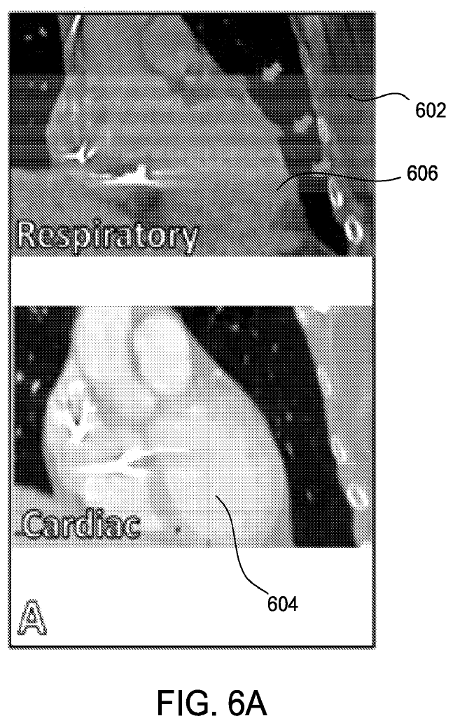

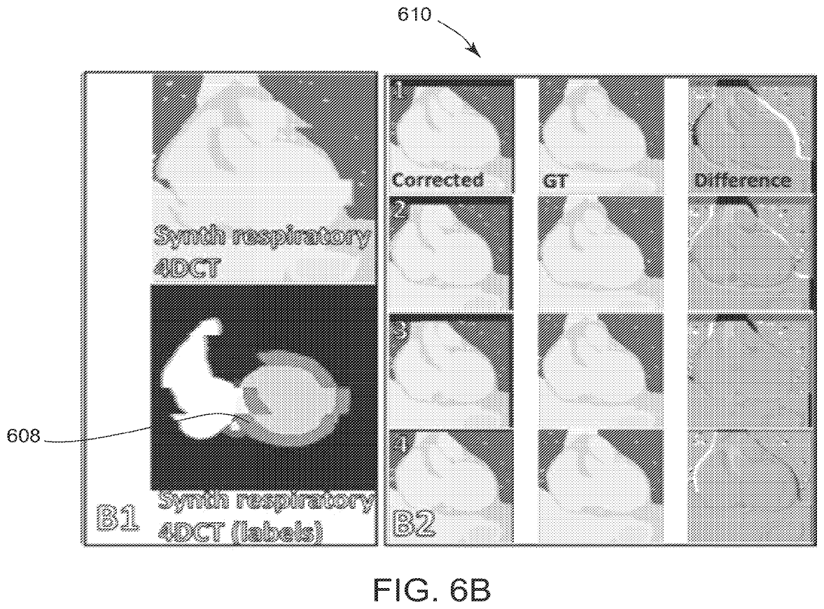

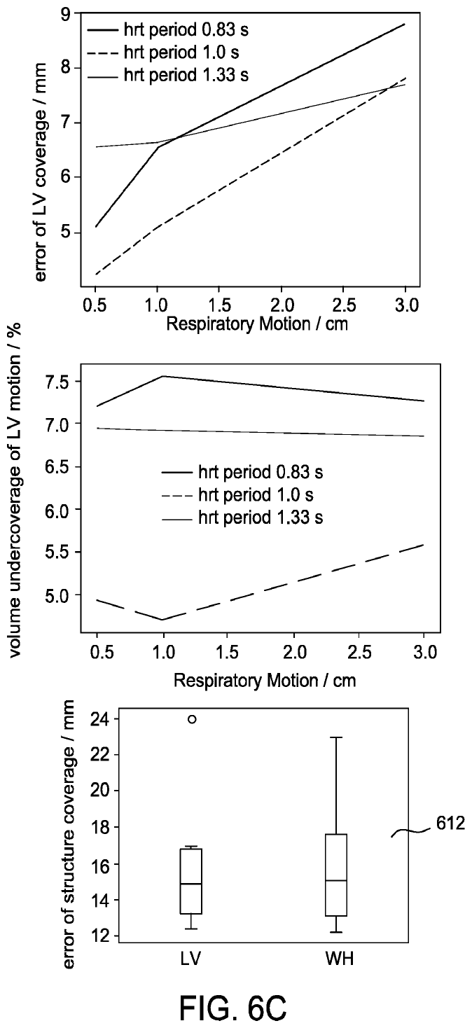

Currently, a respiratory 4DCT captures the total motion of the heart to form a motion envelope or ‘internal target volume’ to encompass the entirety of motion. The issue with this approach is that currently available respiratory 4DCT is not designed to accurately measure heartbeat, which results in motion artifacts. FIGS. 6A and 6B show such artifacts and results of simulations in an anthropomorphic computational phantom, where the ground truth is known. The phantom may be referred to as an XCAT phantom. The phantom was used to simulate respiratory 4DCT acquisition of heartbeat during free-breathing, which showed current respiratory 4DCT methods may result in errors up to the magnitude of breathing motion (>2 cm) and up to 17% volume under coverage of the target (FIG. 6C). For context, typical safety margins in cardiac radioablation are 3-5 mm, which may result in a geographic miss of the target due to this error in target design during treatment planning.

FIGS. 6A-6C show respiratory 4DCT alone does not accurately measure heart motion. In FIG. 6A, an image 602 of respiratory 4DCT and an image 604 of cardiac 4DCT are shown. Artifacts 606 are clearly visible as stack transitions on the respiratory 4DCT, whereas the lateral wall is artifact free in the cardiac 4DCT. In FIG. 6B, 4DCT acquisition was simulated using the XCAT phantom, showing similar stack transition artifacts as the clinical image. Left ventricle contour 608 is highlighted in red. Panels 610 are frames 1-4 from a prototype SCARAB-4D algorithm (‘corrected’) show good agreement with ground truth (‘GT’) phantom data. Simulations using XCAT show conventional respiratory 4DCT underestimates heart motion up to 9 mm and 8% volume for reproducible breathing and up to 24 mm and 17% volume for variable breathing. In plot 612, LV stands for Left ventricle, WH stands for whole heart

There are several options to bring joint cardiac/respiratory 4D imaging into the clinic and reduce the errors in respiratory 4DCT. Modern cardiac CT scanners have sufficient longitudinal coverage (>160 mm) to cover the entire heart and motion range in a single table position. Multiple fast ‘frames’ may be acquired to construct a motion pattern representative of both breathing and heartbeat, although this would result in very high patient imaging dose. However, such cardiac scanners are not capable of being used as CT simulators, as the scanners do not have sufficient bore size, capture a smaller field of view (300 mm) of images which truncates the patient and thus cannot be used for dose calculations, and do not have CT simulation software required for radiation therapy. Furthermore, a larger scan range (˜200 mm) is typically desirable to capture position and motion of surrounding organs at risk such as lung and stomach. Another option is cine (volumetric) MRI, but cine MRI does not currently achieve a 3D frame rate high enough to accurately measure heartbeat during free breathing, and there are challenges in calculating dose on MRI due to lack of electron density information, low resolution, and spatial distortion. Further, nearly ubiquitous presence of metal objects (i.e., implantable cardioverter defibrillator (ICD)) limits MRI use as the sole source of treatment planning information.

Development and evaluation of a new algorithm SCARAB-4D, Simultaneous Cardiac And Breathing motion correction from 4DCT (FIGS. 7A-7C) is described, where separate cardiac and respiratory 4DCT scans are combined, enabling estimation of the full motion pattern of the heart and substructures. The goal with SCARAB-4D is to reduce the error in measuring heart motion to be within 3 mm, the typical safety margin used for cardiac radioablation. SCARAB-4D is designed to fulfill the needs for cardiac radioablation planning in the community. It is a post-processing technique which combines separate cardiac 4DCT and respiratory 4DCT scans, each acquired as per clinical standard of care, and therefore requires no changes to the scan parameters or scanner itself. CT is used because CT is the basis for radiation dose calculation and planning in all thoracic patients.

SCARAB-4D will generate a cardiac motion model from a high quality cardiac 4DCT scan, and then fit the model to each acquired phase and ‘stack’ of slice positions on the respiratory 4DCT to generate a simultaneous cardiac and respiratory motion estimate.

### Section A.3.2. Cardiac Imaging Availability for Radiation Therapy Planning

Major challenges integrating imaging information into cardiac radioablation planning include the availability of specific types of imaging and time between imaging studies. Many clinics do not have cardiac imaging available in the radiotherapy department where radiotherapy-specific imaging is acquired. It is therefore the SCARAB-4D technique is broadened to be applicable to clinics without radiotherapy-specific cardiac imaging capabilities. To do so, cardiac 4D imaging acquired on a different day or time is used to create the motion model in SCARAB-4D. One possible scenario in the community is the acquisition of a cardiac 4DCT or cardiac MRI scan in the cardiology or radiology department, followed by a planning CT (with respiratory 4DCT) on a radiation oncology CT simulator, typically on a different day. In this scenario, there may be days or weeks between acquisition of the diagnostic cardiac imaging and respiratory 4DCT for radiotherapy simulation. Furthermore, some clinics may not have cardiac 4DCT available, and have cardiac MRI instead. Thus, the algorithm developed is capable of using a multi-modality cardiac motion model.

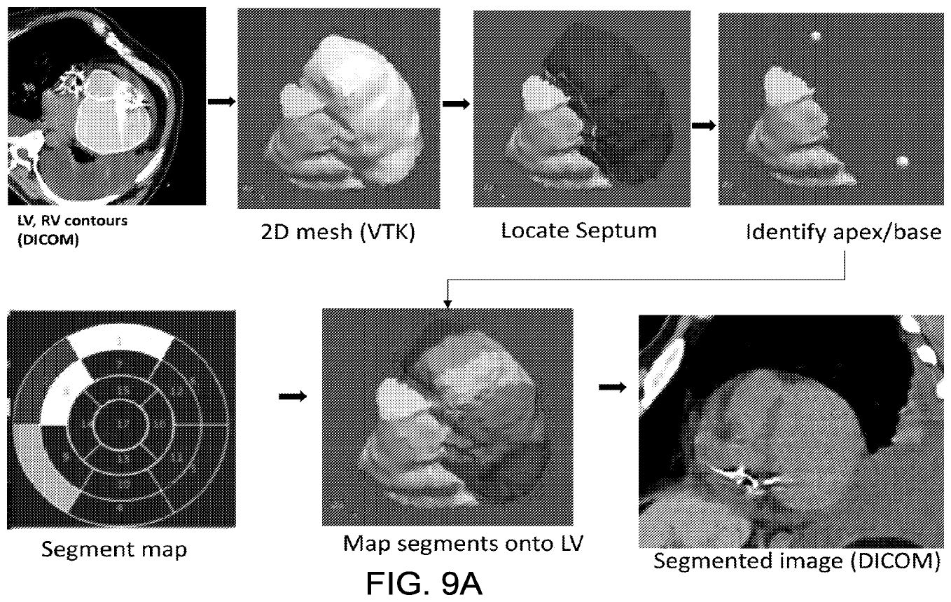

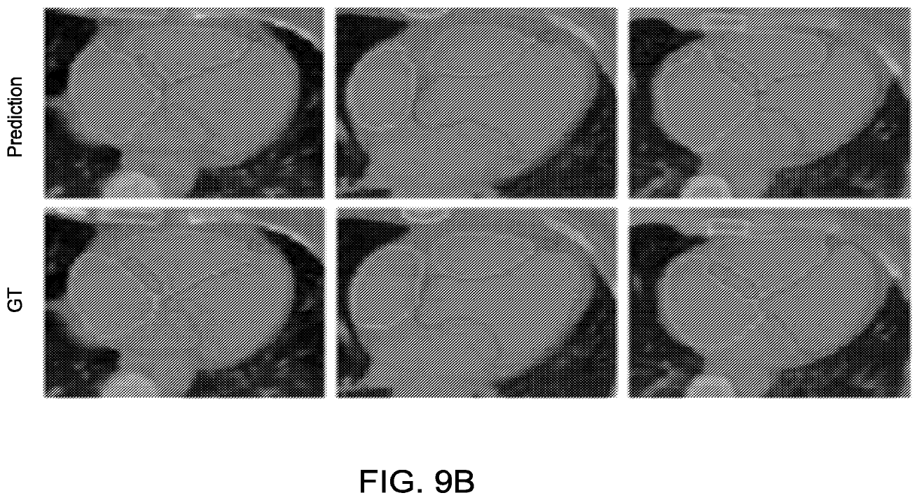

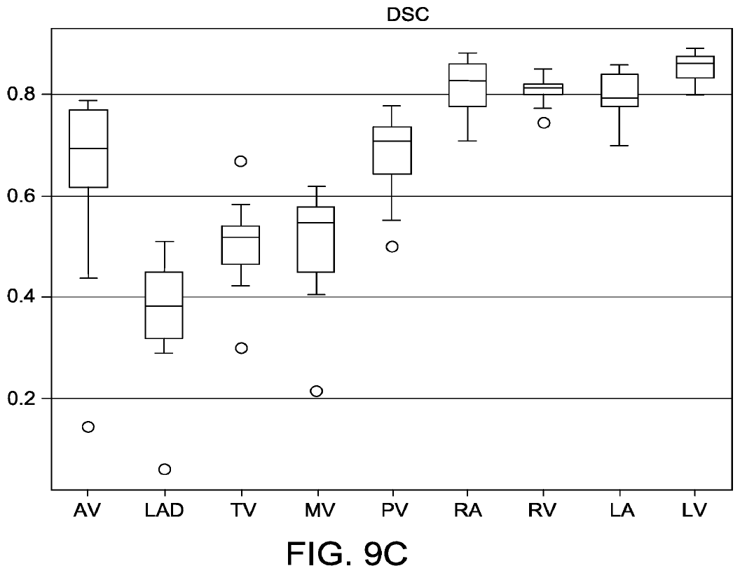

To broaden applicability of the SCARAB-4D technique, the cardiac 4D imaging acquired on a different day may be used as the underlying heart motion model. However, anatomical deformations and volume changes (e.g., due to change in volume status) along with imaging modality differences are managed. To do so, a technique combining deep learning-based autosegmentation of anatomical heart features with a feature-based deformable image registration algorithm is used to accurately register cardiac and respiratory 4DCT images acquired on different days, and to bring in multi-modality imaging such as MRI, where images acquired by a different modality is registered with 4DCT images. The data demonstrate that deep learning is feasible on contrast and non-contrast CT for segmenting the heart chambers (see FIGS. 9A-9C described later).

Segmentation of the left and right ventricles may then be used to extract anatomical keypoint information and fit a 17 segment model onto the epicardial surface. Along with endocardial keypoint anatomy, this information may be used to guide an anatomical keypoint-based deformable registration algorithm. The segment model-based registration approach will fit the cardiac model onto the anatomy of the day of the respiratory 4DCT scan, which will then be used to reconstruct the heart/respiratory motion of the heart using SCARAB-4D. This approach may be used to fuse any type of multi-modality imaging data (e.g., CT, MR, PET, SPECT) with the planning CT.

### Section A.3.3. Current Dosimetry and Planning Techniques do not Account for Dynamic Nature of Delivery

Currently, radiation dose calculations during planning assume the anatomy is static. Moving anatomy such as the heart perturbs the dose distribution, resulting in a discrepancy between the planned and delivered dose. This results in uncertainty and error in the estimation of target radiation dose and also in estimation of dose to normal organs. For example, dosimetric errors up to 20% have been observed in the thorax for static compared to dynamic dose. There are several implications of this error. First, a safety margin known as an internal target volume (ITV) is used to address the effect of such motion but results in a larger target and therefore more normal tissue irradiated. Second, since the true dose delivered is not known, the estimated planned dose is typically used in radiation response models as an independent predictor of toxicity. For cancer treatments, better estimation of true dose results in better outcomes and toxicity prediction models. However, this is untested in cardiac radioablation patients.

In general, 4D dose calculation methods are available in research systems. However, there are no methods available to calculate dose to the heart and adjacent organs due to both breathing and heartbeat. 4D dose calculation methods are adapted to work with the SCARAB-4D motion models described above in order to estimate the true delivered dose to the target, heart substructures (such as chambers, valves, and coronary arteries), and other risk structures such as stomach and lungs. Because CT images have electron density information, 4D CT images are used to calculate radiation dosage, based on the relationship between the image intensity and electron density. The 4D dose calculation provides the dose at discrete phases of motion, due to heartbeat and/or respiration, which may be summed or averaged across phases to provide a more accurate representation of the delivered dose in the presence of motion. Further, because corrected respiratory images using the systems and methods described herein have increased image quality and reduced artifacts, 4D dose calculation based on the corrected respiratory images has increased precision. Delivered dose will then be used to establish appropriate safety margins (ITV and analogous planning at risk volumes for organs at risk). The delivered, cumulative dose is used to improve cardiac radioablation outcome and toxicity models.

### Section A.4. Broader Impact

In addition to solving problems in treatment planning for cardiac radioablation, the results of this project may have wider ranging impact. In cancer radiotherapy, the systems and methods described herein of improved respiratory 4DCT may enhance imaging for planning in the thorax (such as lung, esophagus, breast), where heart-induced artifacts affect target and organ at risk definition and dosimetry. That is, systems and methods described herein may be used to enhance planning for therapy of thoracic anatomies other than the heart. The cardiac models described herein may also be useful in improving on-treatment imaging such as cone beam CT and/or onboard MRI, where motion model-based image reconstruction may result in faster and better quality images to help guide patient setup and plan adaptation.

### B. Technical Effects

Cardiac radioablation is an emerging modality for treatment of VT. At least one technical effect of the systems and methods described herein include:

Development of strategies for dealing with combined respiratory and cardiac motion. To date, there are no methods for accurate imaging of the heart under free breathing for radiotherapy planning, particularly with CT. Methods described above are developed for this purpose, which may be deployed on existing CT infrastructure already present in radiotherapy departments.

Development of multi-modality cardiac image registration strategies. Using ventricular segment models, a hybrid model-, intensity-, and contour-driven deformable registration algorithm is used to align multi-modality cardiac imaging to radiotherapy simulation CT.

Estimation of cumulative dose in the cardiac radioablation setting. All clinical cardiac radioablation studies to date use static dose estimates. Here, cardiac and respiratory motion is incorporated into dose calculation and provides an estimate of cumulative dose in consideration of motion.

Establishment of cardiotoxicity outcomes models for cardiac radioablation. There are no outcomes models for cardiac radioablation. The tools developed here will be a first step in establishing such models.

### C. Approach

### C.1. Develop and Evaluate a 4D Computed Tomographic Imaging Technique Capable of Accurately Measuring Simultaneous Cardiac and Respiratory-Induced Heart Motion.

### C.1.1. Strategy/Methods

SCARAB-4D CT correction algorithm. The goal is to develop an approach to correct respiratory 4DCT scans to better measure the shape and motion of the heart and adjacent anatomy during the scan acquisition, so that better estimates of target volume and motion may be used in target definition. SCARAB-4D will need a respiratory 4DCT that will be corrected, and an ECG-gated cardiac 4DCT, where each may include 8-10 frames of temporal data. Only the reconstructed scans are needed, which are frames of images at different cardiac phases. That is, SCARAB-4D does not need the raw projection data, or data or images output after a CT scan before being rebinned to different cardiac phases. This is an advantage because SCARAB-4D may be deployed without modifications to the scanner or reconstruction algorithms and is therefore applicable with existing scanners and retrospective data.

FIGS. 7A-7C show the general strategy of the SCARAB-4D algorithm to correct heart motion measurement errors from respiratory 4DCT. FIG. 7A is a schematic diagram of SCARAB-4D. A cardiac motion model is built from a separate cardiac 4DCT using deformable registration. Stack transitions are estimated and stacks of 4D frames are extracted from the respiratory 4DCT. The cardiac motion model is fit to each stack to identify the cardiac phase best associated with the stack. Stack to model registration fits the model to each stack, then the stacks are re-connected to form the corrected respiratory 4DCT. FIG. 7B is a schematic diagram of groupwise registration. All frames are registered simultaneously to a common reference frame using a spatiotemporal transform, hₜ(x), to reduce error in estimating motion fields. An example spatiotemporal transform is a parameterized transform such as a b-spline transform where b-splines are used to parameterize the spatial transformation in the spatial dimensions as well as the temporal changes in the dimension of time. FIG. 7C is a schematic diagram of artifact-weighted groupwise registration. A weight map is generated by detecting outliers (image intensity inconsistencies) across the temporal frames at the same spatial location. Here, a breathing artifact 703 shows as a ‘floating’ piece of the diaphragm. In the weight map, pixels corresponding to artifact 703 have a smaller weight than pixels other than artifact 703. Estimated Motion fields 705 are used to deform the deform or align the images with a reference image. The weight map is then used to suppress artifacts in the deformed, registered image, where it may be seen that the outlier ‘floating’ piece does not contribute to the final reconstructed image 702. As shown in image 704, artifact 703 is significantly reduced.

In the example embodiment, first, a cardiac-only motion model is generated from the cardiac 4DCT with the patient in breath hold to mitigate respiratory motion. Briefly, a groupwise deformable image registration algorithm is used to align each frame of the cardiac 4DCT to a selected reference frame (shown in FIG. 7B). A reference frame may be either a quiescent frame such as end diastole, or the motion-averaged mean image. The end result is a self-registered image where each frame is registered to the reference, and the resulting transform maps tissue positions through the cardiac cycle. Next, the respiratory 4DCT to be corrected is separated at the stack junctions (see FIG. 7A) into sets of individual stacks, where each stack was reconstructed during the same scanner rotation. A stack contains the entire axial image field of view, and in a modern CT simulator is approximately 2 cm of data in the scan direction. Stack junctions may either be identified retrospectively from the reconstructed image knowing the acquisition parameters and the respiratory signal used to sort the projection data into phases. For each stack, the appropriate phase of the cardiac motion model is selected. If the respiratory 4DCT was acquired with simultaneous ECG, the ECG signals may be used in selection of the appropriate cardiac phase. If the respiratory 4DCT was not acquired with simultaneous ECG, then a registration strategy will be used in estimating the respiratory phase and position from the respiratory signal acquired during respiratory 4DCT acquisition. For example, the stack will be rigidly registered to each phase of the cardiac motion model. The resulting phase with best fit (e.g., by image similarity) will be selected as the cardiac phase for that stack.

After the cardiac phase of each stack is selected, each stack for each respiratory phase is then registered individually to the selected cardiac phase image from the motion model, which may be referred to as “model to stack registration”. Because the cardiac 4DCT was acquired at the same time as the respiratory 4DCT, minimal change occurs in heart shape and volume between these two scans. For this reason, a rigid registration should be suitable for matching the cardiac model to the stack. Finally, the registration result for each stack is used to map the appropriate cardiac phase image onto the respiratory 4DCT scan, for each stack and each respiratory phase. The final result is a motion model-reconstructed estimate of heart shape and position during the respiratory 4DCT scan.

SCARAB-4D (FIG. 7A-7C) was implemented on the XCAT computational anatomy motion phantom, which has programmable heart and respiratory motion, with a known ground truth motion pattern. For this phantom study, results are shown in FIGS. 6B and 6C. 4DCT correction systems and methods disclosed herein achieved reasonably accurate results in phantom (FIG. 6B, Hausdorff distance to ground truth 3.7+/−2.9 mm) for breathing motion simulations varying from 5 mm to 15 mm of maximum heart motion. In the original (uncorrected) 4DCT, the Hausdorff distance to ground truth was 6.7+/−4.3 mm for this example of heart motion, showing that our prototype SCARAB-4D improved accuracy in measuring heartbeat and breathing-induced motion by 3 mm, on average. Further studies (FIG. 6C) investigated the effect of various amounts and periods of motion on accuracy of the uncorrected 4DCT scan. These results showed that the uncorrected 4DCT scan underestimated heart motion by 9 mm and 8% volume for reproducible breathing and up to 24 mm and 17% volume for the more realistic case of variable breathing.
Study 1.1: Selection of cardiac motion model registration algorithm. Several registration approaches are tested for building the cardiac motion models, including existing groupwise packages such as elastix, which is based on a b-spline transform. The advantages of this approach are speed and a smooth transform both spatially and temporally. The disadvantage of the b-spline transform is that it may be challenging to model complex deformations such as sliding or torsion as is present in the heart. Thus other groupwise algorithms are also tested, such as Demons and LDDMM, which allow more complex transforms. For each registration algorithm, a model is built for the 18 available same-day cardiac 4DCT scans in the database (see below). Experts in cardiac anatomy will oversee annotation of at least 50 point landmarks in all phases at known anatomical landmarks on the endocardial surface. Approximately 50 landmarks will be sufficient based on the size of the volume. Existing contours of the left and right ventricle epicardial surfaces are also used. Using standard methods to assess registration accuracy, landmark error and surface error (Dice, mean surface distance), will be tabulated for each model and each registration algorithm. The best performing registration approach will be selected for the remainder of cardiac motion measurements.

Available Datasets. Two datasets are available for this work, one from 20 patients enrolled on the ENCORE-VT trial and another from 18 patients treated off-label afterwards. A total of over 100 4DCT and dynamic MRI images are available between the two datasets. Each patient has respiratory 4DCT and cardiac 4DCT. In the first dataset, cardiac 4DCT was acquired at a separate time on a separate, cardiology CT scanner. For the second dataset, of 18 patients, we had acquired a CT simulator (Siemens Confidence RT Pro) in the Radiation Oncology department with both respiratory and cardiac gating capabilities. This second group of 18 patients have cardiac 4DCT and respiratory 4DCT acquired during the same session on the same day on this scanner, without the patient moving between scans. Approximately half of the patients in each dataset have dynamic cardiac MRI available as well, and all patients have electrocardiogramaging (ECGI) available if needed.
Study 1.2: Development of SCARAB-4D algorithm. This study will develop and evaluate the SCARAB-4D algorithm. The major development challenge is the model to stack registration process. Because the stacks are relatively ‘thin’ (˜2 cm in the scan direction), image quality issues may cause direct registration of the whole cardiac model to this thin stack to be challenging. Image quality issues may include metal artifact (due to the ICD leads and possible other implanted devices), noise, and to a lesser extent residual motion blurring due to cardiac motion during a single cardiac phase.

To address these challenges, a groupwise artifact-reduction algorithm is used. Briefly, groupwise registration inherently is robust to artifact as all frames of the cardiac 4DCT are simultaneously considered in the registration, and therefore preferentially registers tissue present in most frames while ignoring artifact which is unique to each individual frame (i.e., metal artifacts appear different in each frame whereas the tissue boundaries are present in all). Contribution in Study 1.2 is to add an outlier detection when the groupwise registration is used to map all frames to a common reference frame, which may be referred as artifact-weighted groupwise registration. Briefly, outlier detection is performed by evaluating the consistency of a region across temporal frames in the registered image (FIG. 7C). Frames with image inconsistency are identified and down-weighted in the deformed image.

This artifact-weighted groupwise approach will be evaluated to determine if it may be used to reduce the impact of artifact on the motion model creation and model to stack registration. For model to stack registration, instead of registering the stack to a single frame from the cardiac model, the artifact-weighted groupwise registration is used to map all frames from the cardiac motion model to the selected cardiac frame, and then perform a groupwise registration of the stack to all of these frames. This process is analogous to the artifact-weighted groupwise registration but will robustly register the stack to the aligned cardiac frames, minimizing the impact of artifact on the model to stack registration.

Evaluation methodology. To evaluate the quality and accuracy of SCARAB-4D reconstructions, both phantom and clinical images will be used. Using the previously described XCAT phantom, a series of ground truth motion patterns will be constructed with various heartbeat and breathing frequencies, motion amplitudes, and heart and patient size, shape, and position variations. A software package has been developed for simulating a 4DCT acquisition (see FIG. 6B), which will be used to simulate 4DCT scans with different scanning parameters (e.g., pitch and breathing period). Using the ground truth phantom data, simulated respiratory 4DCT acquisitions will be produced. Ground truth cardiac 4DCT will also be generated. The reconstruction algorithm will then be used to reconstruct the respiratory 4DCT using the cardiac model. Knowing the ground truth motion pattern, the reconstructed results may be compared directly with the ground truth. In this manner, measurements may be performed such as spatial accuracy (distance to agreement in the organ or partial organ surfaces) and overlap (Dice similarity) between the reconstructed anatomy and ground truth, for heart, left ventricle, and organs at risk such as stomach and esophagus.

In addition, clinical images will be used to estimate performance of SCARAB-4D. The same epicardial contours and landmarks as in Study 1.1 will be used for validation here. These data will be used to establish the ground truth in the clinical images. Landmarks may be used directly to assess spatial accuracy using existing techniques. The contours of each stack will be used to estimate registration accuracy based on distance to agreement and overlap between the model-reconstructed contours and physician-delineated contours.

Power analysis. From preliminary data, the spatial error between the original respiratory 4DCT and ground truth cardiac and respiratory motion is estimated as 16.1 mm+/−4.3 mm while our target goal is 3 mm. To achieve a power of 80% and a level of significance of 5% (two sided), for detecting a mean of the differences of 13.1+/−4.3 mm (paired study) 5 patient datasets are needed, each with a same day respiratory and cardiac 4DCT. These data are already available from our patient database which contains 18 patients with same day scans.
Study 1.3: Prospective evaluation of SCARAB-4D. To evaluate the accuracy of the corrected respiratory 4DCT scan, respiratory 4DCT and oversampled cardiac 4DCT in the same patient will be collected. For sample size calculation, the same preliminary data as in Study 1.2 above will be used. However, as these preliminary data are from simulations, the study size will be doubled to 10 patients. Patients being simulated for cardiac radioablation, typically 2-3 per month, will be considered for enrollment on this imaging study. Unlike existing 18 patient database, here cardiac 4DCT will be collected at both end of inhalation and end of exhalation to model the extent of both respiratory and cardiac motion. These paired cardiac scans will provide the range of both heartbeat and breathing motion, enabling construction of a ground truth motion envelope, with minimal artifact. Respiratory 4DCT will also be acquired, and SCARAB-4D will be used to correct the respiratory 4DCT with the end inhale cardiac 4DCT. The resulting motion envelope from the correction will be compared to the ground truth, using the same approach as in Study 1.2

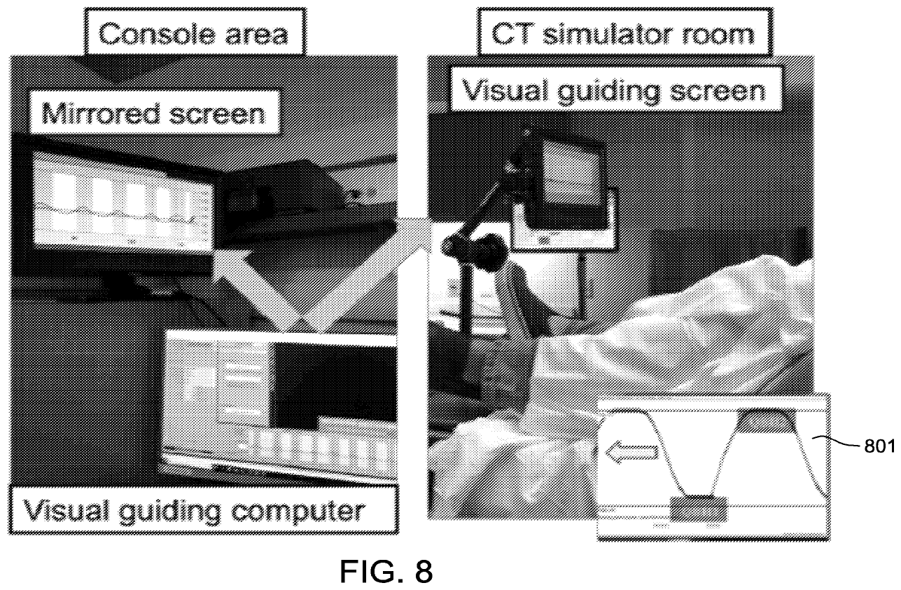

Patient respiration may vary breath to breath, and voluntary breath hold at end exhalation/end inhalation may not correspond directly with the same motion range during free breathing. To ensure that the respiratory motion acquired during respiratory 4DCT matches that during patient end inhale/end exhale breath hold during cardiac 4DCT acquisition, a visual feedback system will be used to assist the patient in performing reproducible free breathing and breath hold maneuvers. FIG. 8 shows an example visual feedback system. A commercial respiration monitoring system (visual guiding computer) is mirrored and projected into the CT simulation room, where the patient views the screen and follows a prescribed breathing or breath hold curve (Insert 801). This visual feedback system will be used to ensure reproducible respiration between the different scans in this study.

### C.1.2. Potential Outcomes, Problems, and Alternatives

A successful outcome will be a final SCARAB-4D algorithm capable of retrospectively reconstructing accurate respiration and cardiac motion from a standard respiratory 4DCT and cardiac 4DCT. The model to stack registration approach may be found not performing with sufficient accuracy, as the model to stack registration will be a challenging registration problem due to the small image size of each stack, the potentially large motion range, and the ubiquitous presence of metal objects. If this main approach fails, methods will be evaluated to further improve the registration accuracy, including 1) selection of a more constrained (e.g., smoother) registration algorithm, 2) try to add more data to the stack by extrapolation or combining stacks from the same cardiac phase, 3) investigate a supervised registration approach such as using phantom and/or clinical data with contours to train a registration algorithm using deep learning.

### C.2. Specific to Section A.2. Develop and Evaluate Multi-Modality Cardiac Motion Models to Manage Day to Day Anatomical Changes.

### C.2.1. Strategy/Methods

The basic concept of this section is to develop a SCARAB-4D algorithm in cases where a cardiac 4DCT is not available during radiotherapy simulation, as assumed in Section A.1. Here, instead, available cardiac 4DCT acquired at a different time, or multimodality cardiac motion data such as dynamic cardiac MRI, will be used. Anatomical variability between days, and differences in image intensity and content between different imaging modalities, will additionally complicate the already challenging model to stack registration process. For this reason, a technique will be used that combines deep learning-based autosegmentation of anatomical heart features with a segment model-based deformable image registration algorithm to accurately register cardiac images to respiratory 4DCT images to perform reconstruction of the respiratory 4DCT scan as in Section A.1. The model to stack registration process will be extended to incorporate the American Heart Association 17 segment left ventricle model to guide the deformation of the cardiac model onto each stack. The performance of the algorithm will be evaluated on existing multimodality imaging data.

The basic structure of the Section A.2 approach is presented in FIGS. 9A-9C. FIG. 9A is a schematic diagram of 17 segment left ventricle to image registration algorithm. The segments included in the 17 segment model include anatomical segments, because the segments are based on anatomy and are anatomical features. Left and right ventricle contours on a MR or CT image are converted to 3D meshes, the septum is identified using an approach of using closest distance between LV and RV, and principal components analysis is used to find the long axis of the LV. Finally, the 17 segment model is mapped onto the LV mesh knowing the location of the anterior and posterior interventricular grooves, and the long axis of the LV. This segmented mesh is then converted back to a 3D image in the same domain as the original image and may be imported into a radiation therapy treatment planning system or image analysis package in DICOM format. FIGS. 9B and 9C show deep learning autosegmentation of CT substructures. Deep learning autosegmentation was trained from 250 planning CTs from patients undergoing thoracic radiation therapy. The results show clinically acceptable performance for the LV and RV (Dice comparison to physician-drawn contours >0.8).

The 17 segment model is mapped onto the left ventricle in each image. Because the segments in this model are based on anatomy (the regions perfused by each coronary artery along with the interventricular grooves), registration of the heart anatomy may be accomplished by mapping the segment model onto each image and then using this model to identify corresponding keypoint anatomy (e.g. interventricular grooves) and interpolate correspondence at other locations. A prototype algorithm has been developed for mapping the segment model onto a cardiac image (FIG. 9A). The left and right ventricle contours are used to identify the interventricular grooves. Principal component analysis of the left ventricle contour is used to identify the long axis of the left ventricle. These two pieces of information are used to map the cone-shaped segment model onto the left ventricle contour, which may then be transferred back to the image space using an existing algorithm.
Study 2.1. Development and evaluation of deep learning autosegmentation of cardiac substructures in CT and MRI. This study is designed to develop and evaluate autosegmentation of the left and right ventricles, as these structures are needed for mapping the segment model onto the left ventricle, as explained above and in FIG. 9A. A deep learning based autosegmentation of the cardiac substructures (ventricles, atria, valves, and left anterior descending coronary artery) has been developed using existing contours in 250 CT scans. Performance was considered excellent for the left and right ventricles, with Dice similarity >0.8 compared to physician-drawn contours in a holdout test dataset of 10 patients (FIG. 9C). In this study, this autosegmentation will be extended to contrast enhanced cardiac MRI and CT, including not only the epicardial ventricle surfaces, but also the endocardial surfaces. In preliminary studies on non-contrast CT, performance in training with 30 patients (with an additional 10 for parameter tuning and 10 for testing) gave similar results as for the entire 250 patient dataset. Therefore, 50 datasets are planned to be collected for each of contrast enhanced CT and contrast enhanced MRI. Physicians will contour the left and right epicardial surfaces of the ventricles and the endocardial surfaces. The derived contours are used as training datasets. Best practices in deep learning autosegmentation will be used, including testing on a held out dataset, using an independent dataset of training for parameter tuning, using cross validation, and using data augmentation such as rotation, zoom, and shift to augment the training dataset. Performance will be quantified in the held-out test dataset (10 each, MRI and CT) using established methods such as distance to agreement and Dice similarity between the autosegmented and physician contours.
Study 2.2. Development and evaluation of segment model mapping to cardiac imaging. This study will evaluate the ability to accurately map the segment model onto the left ventricle in cardiac CT and MRI. Development of the algorithm will be based on the prototype shown in FIGS. 9A, which have been developed for use in contrast and non-contrast radiotherapy CT simulation. Here, the systems and methods will be extended to cardiac MRI. Using the autosegmented and physician-drawn contours in Study 2.1, the accuracy will be investigated in mapping the segment model onto the image. Physicians will manually segment the CT and MRI images in the test (holdout) datasets from Study 2.1 to the 17 segment model using a cardiac atlas. The automated segments using image mapping algorithm developed here will then be compared to the manual method for accuracy (see FIG. 9B).
Study 2.3. Evaluation of SCARAB-4D using cardiac imaging motion models. In this study, the ability of SCARAB-4D to reconstruct respiratory 4DCT is evaluated using a cardiac motion model from different days and different modalities and accurately measure cardiac and respiratory motion of the heart and organs at risk. The major challenge of this study is to develop and evaluate an accurate model to stack registration process. Because the cardiac motion model will possibly have anatomical (shape and volume) differences as it may be acquired on a different day or time than the respiratory 4DCT desired to be reconstructed, a rigid registration strategy as in Section A.1 may not be used. Instead, here, the segment model will be used to guide the registration process.

First, the segment model will be applied to each frame from the dynamic cardiac CT or MRI scan to build a motion model. In each frame, the left and right ventricles will be autosegmented and the model applied as described above. Then, the segment model in each frame will be used to identify the epicardial surface correspondence between frames. A point-based registration using iterative closest points will be used to register the corresponding segments.

Next, the segment model will also be applied to the respiratory 4DCT. This will result in a segment model with discontinuities at the stack boundaries, but with a complete segment model on the surface. A hybrid registration approach will be used, which jointly registers the cardiac model (epicardial segments, endocardial surface contour, and image intensity) to each stack in the respiratory 4DCT (epicardial segments, endocardial surface, and image intensity). The endocardial surface typically has more features than the epicardial surface, which tends to be smooth. Hence, the endocardial surface contour is directly used in the registration whilst the epicardial surface is represented by the segment model.

Evaluation methods and power analysis. The reconstructed respiratory 4DCT will be evaluated using similar methods as in Study 1.2, by comparison to physician-drawn contours and to annotated landmarks. The dual end inhale/end exhale cardiac 4D-generated ground truth from Study 1.3 will be used to assess performance in this section. In addition, the results of reconstruction using the multimodality cardiac model will be compared to that from Section A.1, where the cardiac model was acquired on the same day. The cohort size is based on a paired non-inferiority comparison between the accuracy of the model-based reconstruction using the methods in this section (Study 2.3 methods) to those using the Section A.1 methods on the day-of cardiac 4DCT based model (Study 1.2 methods). The expected mean error in model reconstruction of left ventricle is 3.7 mm+/−2.9 mm, and the inferiority margin is 2 mm, resulting in a cohort size of 18 patients. These data are available already.

### C.2.2. Potential Outcomes, Problems, and Alternatives

The major outcome is a functioning SCARAB-4D algorithm to generate accurate measures of respiratory and cardiac of heart and/or other organs using multi-modality or different-day cardiac imaging. Preliminary results with deep learning demonstrate strong performance in segmenting the ventricles in non-contrast CT. This model should be adaptable to cardiac MRI segmentation. If the basic strategy of transfer learning does not result in sufficient accuracy, style transfer (MR to CT intensity conversion) will be used as in the alternative solution in Section A.1. Other alternatives include retraining the model on MRI data, which may be time intensive. Finally, manual contours may be used directly, although the clinical process would be labor intensive. If the registration approach is not sufficiently accurate, conversion of MR to CT intensity will be evaluated. Then, an approach similar to the Section A.1 approach may be used to register a synthetically-generated cardiac 4D model to the respiratory 4DCT.

### C.3. Estimate the Planned, Cumulative Dose to Heart Substructures for Improved Target Dosimetry and Reduced Toxicity Risk.

### C.3.1. Strategy/Methods

The systems and methods use the developed cardiac motion models along with the reconstructed respiratory 4DCT scans to better estimate the cumulative per-voxel dose to the heart and organs at risk during radiation delivery. The reconstructed 4DCT scans will be used to estimate motion during delivery and measure the cumulative dose. Here the ‘4D dose calculation’ method will be extended from breathing motion to incorporate heartbeat-induced motion.

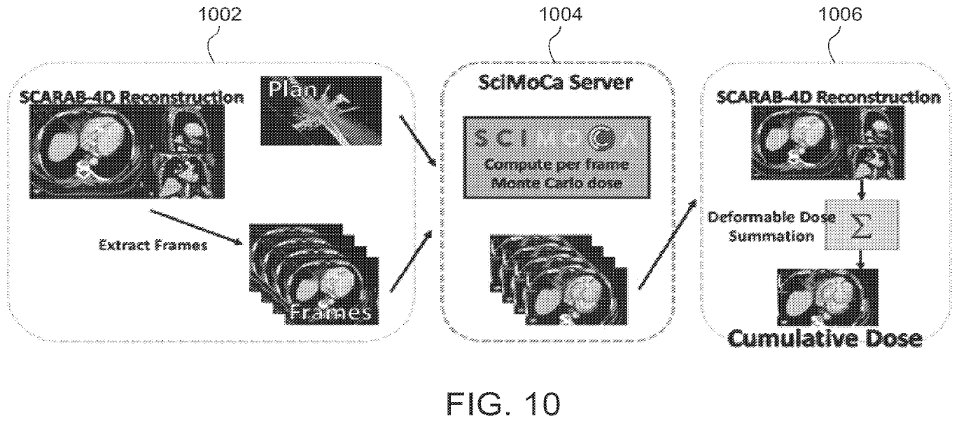

FIG. 10 is a schematic diagram on computation of cumulative dose. In diagram 1002, individual temporal 3D frames are extracted from the SCARAB-4D reconstruction and passed with the radiation plan to the SciMoCa compute server 1004. SciMoCa performs a Monte Carlo dose calculation on each frame, resulting in the 3D per-frame dose, which is passed back to the calling system. The known voxel trajectories in each frame from the SCARAB-4D reconstruction are used to deform each per-frame dose to a reference frame, which are summed to arrive at the cumulative dose (see diagram 1006).

The corrected respiratory 4DCT scan for each patient measures the shape and position of the heart, target, and organs at risk during scan acquisition. The transformation linking the cardiac motion model to each ‘frame’ in the SCARAB-4D reconstructed respiratory 4DCT is derived, as it is inherently measured by the model to stack registration. Dose calculated on any frame of the respiratory 4DCT scan may be linked to a reference frame, and the transformation used to map per-frame dose to the reference. Once individual doses from each frame are mapped to the common reference frame, the doses may be summed to measure the cumulative delivered dose from the treatment.

For the dose calculation, a state of the art dose calculation algorithm will be used to ensure highly accurate and high throughput dose calculation. There may be several hundred frames of temporal data, each frame having a 3D image of the heart and surrounding anatomy, on which a treatment plan dose calculation is performed. It will be challenging to do this work in a clinical treatment planning system, which is designed for dose calculation on a single 3D image. Instead, a commercial-grade research dose calculation engine, SciMoCa, will be used. SciMoCa is a standalone Monte Carlo dose calculation service available in a client-server architecture. Each 3D frame may be submitted along with the radiation plan to a dose calculation server, which computes and returns the radiation 3D dose. SciMoCa has been shown to have accuracy indistinguishable from a state of the art commercial dose calculation algorithm. Once returned from the server, the individual 3D dose images at each frame are deformed using the SCARAB-4D reconstruction and summed to generate the cumulative dose.
Study 3.1. Evaluation of accuracy of motion model-based dose accumulation. In this study, dose accumulated manually at annotated anatomical landmarks and contour boundaries will be used as ground truth to evaluate the accuracy of the motion-model accumulated dose. Annotated landmarks and some contours (left ventricle, right ventricle, whole heart, and organs at risk) are available from Studies 1.1 and 1.2. In addition, the manually contoured training data from Study 2.1 will be used to evaluate dose to the remaining chambers, valves, and coronary arteries. If the deep learning algorithm in Study 2.1 results in improved performance for small structures over the preliminary results in FIG. 9A-9C, then these autosegmented contours will be used instead. Finally, the clinically-treated target volume will be delineated on the reference frame. The dose at each landmark, in each frame, will be measured and the cumulative dose summed by hand at these landmarks. The same procedure will be followed for the boundary of each contoured structure. Note that it may not be possible to directly identify the corresponding points on the boundaries of a contour. Thus, using contour boundaries for validation may only be performed by averaging over the entire boundary, instead of validating individual point cumulative dose. Therefore this combination of contour boundaries and landmarks will be used as ground truth.

There will be three sets of doses and dose-volume metrics (e.g., the volume of an organ receiving at least a certain dose, which are widely used as surrogates for target control and toxicity in radiotherapy) computed and compared. As described above, the cumulative dose at the landmark positions will serve as ground truth. The cumulative dose as estimated from the reconstructed respiratory 4DCT, extracted at the corresponding landmark locations in the reference frame, will be tabulated. Finally, a static dose, which is the calculated radiation dose on the reference frame only (the clinically-used approach currently), at the landmark locations, will be tabulated to serve as a comparison of the clinical standard of care. The cumulative dose error will be evaluated, which is the difference in cumulative dose at the landmark locations between the SCARAB-4D reconstructed 4DCT approach and the actual cumulative dose at the landmarks. The static dose error will also be evaluated, which is the difference in cumulative dose, static dose calculation compared to the landmark-calculated dose. These dosimetric errors will be performed voxel to voxel, and also for a variety of clinically meaningful dose-volume statistics to target, heart substructures, and organs at risk such as stomach, esophagus, and spinal cord.

Power analysis. The primary endpoint of the study is error, model-reconstructed dose vs. landmark-calculated dose. Because dose is directly related to registration error, the power calculation for Study 1.2 may be applied here, resulting in a minimum of 5 patients. However, the results in all 28 patients will be evaluated with respiratory and cardiac 4DCT (18 patients from existing data and 10 patients from the Study 1.3 prospective imaging study).
Study 3.2. Estimation of dosimetric safety margins. Study 3.1 will provide an estimate of the cumulative, delivered dose to each patient's heart substructures, target volume, and organs at risk. These results, coupled with the patient's motion models, will allow safety margins to be established for various motion management strategies.

Currently, the most commonly used method for managing motion is to design a ‘motion envelope’ or internal target volume from the conventional respiratory 4DCT to compensate for heartbeat and respiratory motion during free-breathing. Using the motion models, a variety of motion management strategies may be simulated, including using this motion envelope (but with improved accuracy using the motion model rather than the uncorrected respiratory 4DCT), delivery during breath hold, and respiratory and/or cardiac gating. Here, each strategy will be simulated, the cumulative dose to the target and organs at risk will be measured, and the appropriate safety margins needed to expand each structure will be determined to ensure that target coverage and organ at risk constraints are not violated.

### C.3.2. Potential Outcomes, Problems, and Alternatives

Systems and methods described herein are used to determine the cumulative dose to the target and organs at risk, and a set of safety margins for various respiration management strategies. These safety margins may then be used by the community as they deploy more sophisticated motion management strategies. One potential risk of the methods is the reliance on the resulting cardiac motion models. Preliminary data demonstrates (FIGS. 6A-6C) that building a cardiac motion model is feasible. Thus, little risk is anticipated with this strategy. However, to reduce interdependence, if the SCARAB-4D work were to fail, accumulation of the entire dose distribution will be changed to a landmark-based assessment of the cumulative dose vs. static dose. The difference in cumulative dose vs. static dose at the landmark points will be assessed. This distribution would estimate the error distribution between static and cumulative dose for each patient, and with enough landmarks distributed throughout the heart and organs at risk, may be used as an estimate of the full (all voxels in the image) error distribution in each patient. This backup strategy would not enable full assessment of dose-volume metrics but would allow estimation of the variability in the dose volume metrics from the landmark-based dose uncertainty.

At least one technical effect of the systems and methods described herein includes (a) correcting motion in respiratory images for radiation planning using a cardiac motion model; (b) correcting motion in respiratory images using cardiac images acquired at different time or with a different modality; (c) reducing artifacts in respiratory images using a weight map; (d) estimating dose based on corrected respiratory images.

Example embodiments of systems and methods of motion correction are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method of correcting motion in respiratory four-dimensional computed tomography (4DCT) images of a subject in radiation planning, comprising:
receiving respiratory 4DCT images, wherein the respiratory 4DCT images were acquired while a subject was free breathing;
receiving cardiac four dimensional (4D) images of the subject, wherein the cardiac 4D images were acquired while the subject was in breath hold;
deriving a cardiac motion model based on the cardiac 4D images, wherein the cardiac motion model includes frames of images of the subject, each frame corresponding to a cardiac phase in a cardiac cycle, each frame of images including motion fields at the cardiac phase;
correcting motion in the respiratory 4DCT images using the cardiac motion model; and
outputting the corrected respiratory 4DCT images.

2. The method of claim 1, wherein correcting motion further comprises:
separating the respiratory 4DCT images into frames of respiratory 4DCT images, each frame corresponding to a respiratory phase in a respiratory cycle; and
for each frame,
determining a corresponding cardiac phase of the frame of respiratory 4DCT images; and
correcting the frame of respiratory 4DCT images using the cardiac motion model at the corresponding cardiac phase.

3. The method of claim 1, wherein the respiratory 4DCT images further include stacks of respiratory 4DCT images, each stack corresponding to a portion of a slice coverage of the respiratory 4DCT images, and correcting motion further comprises:
for each stack,
determining a corresponding cardiac phase of the stack of respiratory 4DCT images; and
correcting the stack of respiratory 4DCT images using the cardiac motion model at the corresponding cardiac phase; and
generating the corrected respiratory 4DCT images by combining stacks of corrected respiratory 4DCT images.

4. The method of claim 1, wherein correcting motion further comprises:
detecting outliers across frames of the cardiac 4D images at the same voxel;
generating a weight map based on the detected outliers, wherein voxels corresponding to the outliers have reduced weights; and
reducing artifacts in the corrected respiratory 4DCT images by downweighing the corrected respiratory 4DCT images with the weight map.

5. The method of claim 1, wherein correcting motion further comprises:
selecting a common reference frame;
registering frames of images in the cardiac motion model to the common reference frame to derive a modified cardiac motion model; and
registering the respiratory 4DCT images to the modified cardiac motion model to derive the corrected respiratory 4DCT images.

6. The method of claim 1, wherein deriving a cardiac motion model further comprises:
separating the cardiac 4D images into frames of cardiac 4D images, each frame corresponding to a cardiac phase;
selecting a common reference frame; and
registering the frames of cardiac 4D images to the common reference frame to derive the cardiac motion model.

7. The method of claim 1, wherein the cardiac 4D images were acquired at a different imaging session or using a different modality from the respiratory 4DCT images, wherein:
deriving a cardiac motion model further comprises:
segmenting the cardiac 4D images into segmented cardiac 4D images having anatomical segments, wherein the anatomical segments correspond to standardized myocardial segments in a standardized segment model; and
deriving the cardiac motion model based on the segmented cardiac 4D images; and
correcting motion further comprises:
segmenting the respiratory 4DCT images into segmented respiratory 4DCT images having the anatomical segments; and
registering the segmented respiratory 4DCT images using the cardiac motion model.

8. The method of claim 7, wherein segmenting the cardiac 4D images further comprises:
detecting anatomical features of a heart of the subject in the cardiac 4D images using a neural network model;
deriving keypoint features based on the detected anatomical features; and
mapping the standardized segment model to the cardiac 4D images to derive the segmented cardiac 4D images using the keypoint features.

9. The method of claim 1, further comprising:
determining radiation dosage based on the corrected respiratory 4DCT images.

10. The method of claim 9, wherein determining radiation dosage further comprises:
for each frame of the corrected respiratory 4DCT images,
calculating a radiation dose corresponding to the frame; and
combining radiation doses across the frames into a cumulative radiation dose in a radiation plan.

11. A radiation planning system, comprising a computing device, the computing device comprising at least one processor in communication with at least one memory device, and the at least one processor programmed to:
receive respiratory four-dimensional computed tomography (4DCT) images, wherein the respiratory 4DCT images were acquired while a subject was free breathing;
receive cardiac four dimensional (4D) images of the subject, wherein the cardiac 4D images were acquired while the subject was in breath hold;
derive a cardiac motion model based on the cardiac 4D images, wherein the cardiac motion model includes frames of images of the subject, each frame corresponding to a cardiac phase in a cardiac cycle, each frame of images including motion fields at the cardiac phase;
correct motion in the respiratory 4DCT images using the cardiac motion model; and
output the corrected respiratory 4DCT images.

12. The system of claim 11, wherein the at least one processor is further programmed to correct the motion by:
separating the respiratory 4DCT images into frames of respiratory 4DCT images, each frame corresponding to a respiratory phase in a respiratory cycle; and
for each frame,
determining a corresponding cardiac phase of the frame of respiratory 4DCT images; and
correcting the frame of respiratory 4DCT images using the cardiac motion model at the corresponding cardiac phase.

13. The system of claim 11, wherein the respiratory 4DCT images further include stacks of respiratory 4DCT images, each stack corresponding to a portion of a slice coverage of the respiratory 4DCT images, and the at least one processor is further programmed to correct the motion by:
for each stack,
determining a corresponding cardiac phase of the stack of respiratory 4DCT images; and
correcting the stack of respiratory 4DCT images using the cardiac motion model at the corresponding cardiac phase; and
generating the corrected respiratory 4DCT images by combining stacks of corrected respiratory 4DCT images.

14. The system of claim 11, wherein the at least one processor is further programmed to correct the motion by:
detecting outliers across frames of the cardiac 4D images at the same voxel;
generating a weight map based on the detected outliers, wherein voxels corresponding to the outliers have reduced weights; and
reducing artifacts in the corrected respiratory 4DCT images by downweighing the corrected respiratory 4DCT images with the weight map.

15. The system of claim 11, wherein the at least one processor is further programmed to correct the motion by:
selecting a common reference frame;
registering frames of images in the cardiac motion model to the common reference frame to derive a modified cardiac motion model; and
registering the respiratory 4DCT images to the modified cardiac motion model to derive the corrected respiratory 4DCT images.

16. The system of claim 11, wherein the at least one processor is further programmed to derive the cardiac motion model by:
separating the cardiac 4D images into frames of cardiac 4D images, each frame corresponding to a cardiac phase;
selecting a common reference frame; and
registering the frames of cardiac 4D images to the common reference frame to derive the cardiac motion model.

17. The system of claim 11, wherein the cardiac 4D images were acquired at a different imaging session or using a different modality from the respiratory 4DCT images, wherein the at least one processor is further programmed to:
derive a cardiac motion model by:
segmenting the cardiac 4D images into segmented cardiac 4D images having anatomical segments, wherein the anatomical segments correspond to standardized myocardial segments in a standardized segment model; and
deriving the cardiac motion model based on the segmented cardiac 4D images; and
correct the motion by:
segmenting the respiratory 4DCT images into segmented respiratory 4DCT images having the anatomical segments; and
registering the segmented respiratory 4DCT images using the cardiac motion model.

18. The system of claim 17, wherein the at least one processor is further programmed to segment the cardiac 4D images by:
detecting anatomical features of a heart of the subject in the cardiac 4D images using a neural network model;
deriving keypoint features based on the detected anatomical features; and
mapping the standardized segment model to the cardiac 4D images to derive the segmented cardiac 4D images using the keypoint features.

19. The system of claim 11, wherein the at least one processor is further programmed to:
determine radiation dosage based on the corrected respiratory 4DCT images.

20. The system of claim 19, wherein the at least one processor is further programmed to determine radiation dosage by:
for each frame of the corrected respiratory 4DCT images,
calculating a radiation dose corresponding to the frame; and
combining radiation doses across the frames into a cumulative radiation dose in a radiation plan.
